(19) United States
(12) Patent Application Publication
(10) Pub. No.: US 2024/0065602 A1
(43) Pub. Date: Feb. 29, 2024

(54) URINE FLOW METER

(71) Applicant: Kohler Co., Kohler, WI (US)

(72) Inventors: William Kuru, Plymouth, WI (US); William Kalk, Sheboygan, WI (US)

(21) Appl. No.: 18/236,723

(22) Filed: Aug. 22, 2023

Related U.S. Application Data
(60) Provisional application No. 63/402,228, filed on Aug. 30, 2022.

(51) Int. Cl. A61B 5/20; A61B 5/00

(52) U.S. Cl. CPC A61B 5/208; A61B 5/6887

(57) ABSTRACT

An apparatus for the measurement of urine flow includes a microphone, a temperature sensor, and a controller. The microphone is configured to detect an audio from the urine flow. The temperature sensor is configured to detect a temperature in a toilet bowl associated with the urine flow. The controller is configured to calculate a urine volume in the toilet bowl based on the temperature in the toilet bowl and calculate a flow rate based on the urine volume and at least one time interval from the audio from the urine flow.

This application claims priority benefit of U.S. Provisional Application No. 63/402,228 (010222-22035A-US) filed Aug. 30, 2022, which is hereby incorporated by reference in its entirety.

### FIELD

The present application relates to a urine flow meter for a toilet, and more specifically in some embodiments, a urine flow meter for flow measurements by combining sonouroflowmetry and thermal uroflowmetry.

### BACKGROUND

The analysis of urine flows may help in diagnosing health conditions. These health conditions may range from poor nutrition to cancer. The health conditions may include enlargement of the prostate gland, bladder cancer, prostate cancer. a urinary blockage, or others. The health conditions may involve infection from viruses, bacteria, parasites or other microbial entities that may be harmful to the human body. For urine flow measurements, existing solutions are bulky. The existing device have a medical or industrial appearance are not often used in the home.

Some example urine flow devices may include pressure transducers, optical height sensors, and color sensors. There are large drawbacks to previous solutions. Pressure sensors require a hole through the vitreous, and optical sensors require a color calibration with an assumption that all urine is the same color, which is not a reliable assumptions. Height sensors require the bowl to be filled to the same point every flush and cannot allow spill-over through the trapway during urination. Height sensors also require information on the bowl surface area as a function of height to complete a calculation. Further, pressure sensors and other sensors that may come in contact with the water and/or urine will naturally become foul to this contact.

### BRIEF DESCRIPTION OF THE DRAWINGS

Exemplary embodiments are described herein with reference to the following drawings, according to an exemplary embodiment.

FIG. 1 illustrates a first embodiment of a flow meter device for a toilet.

FIG. 2 illustrates an example block diagram for the flow meter device of any embodiment.

FIG. 3 illustrates a second embodiment of a flow meter device for a toilet.

FIGS. 4A and B illustrate an example uroflowmeter calculation for the flow meter device.

FIG. 5 illustrates an example sonourflowmeter calculation for the flow meter device.

FIG. 6 illustrates a third embodiment of a flow meter device for a toilet.

FIG. 7 illustrates a fourth embodiment of a flow meter device for a toilet.

FIG. 8 illustrates a fifth embodiment of a flow meter device for a toilet.

FIG. 9 illustrates a sixth embodiment of a flow meter device for a toilet.

FIG. 10 illustrates a seventh embodiment of a flow meter device for a toilet.

FIG. 11 illustrates an example controller for any of the disclosed embodiments.

FIG. 12 illustrates an example flow chart for the controller of FIG. 11.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

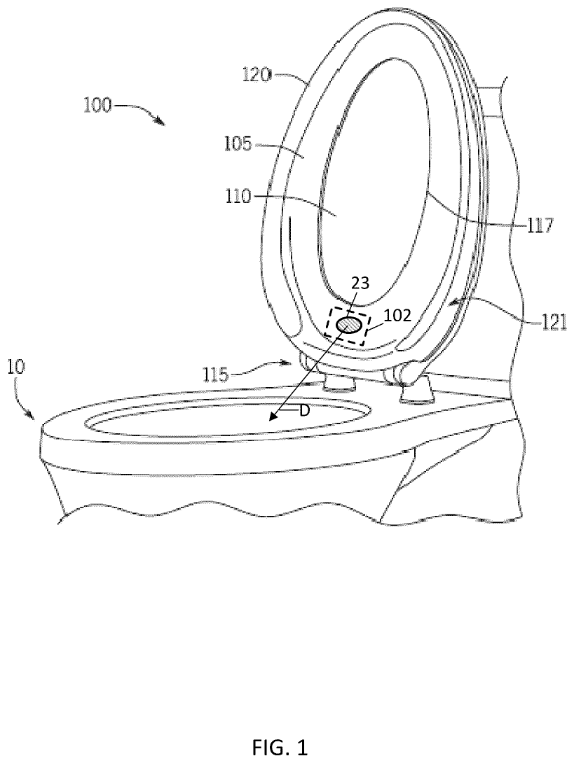

FIG. 1 illustrates an exemplary embodiment of a toilet seat system 100 operatively coupled to a toilet 10. FIG. 1 shows the toilet seat system 100 in a fully lifted configuration wherein both of the lid 110 and the seat 105 are rotated upward about the hinge assembly 115. In various embodiments, toilet 10 may be a one-piece toilet (i.e., tank and base/pedestal are integrally formed) or a two-piece toilet (e.g., tank is separately formed from and connected to a base/pedestal), as well as being either a wall-hung toilet or a floor mount toilet (as shown). In various embodiments, toilet 10 may be siphonic, gravity-fed, pressure-assisted, dual flush, double cyclone, waterless, or any other toilet type known in the art. The toilet seat system 100 includes a seat 105 and a lid 110, which are rotatably engaged and coupled to the toilet 10 via a hinge assembly 115 at a location 15, which is shown rearward of the bowl opening. The toilet seat system 100 includes a flow meter device 102, which may be housed inside a frame of the seat 105. One or more sensors of the flow meter device 102 may detect properties of the ambient environment, properties of water in the toilet 10, properties of urine in the toilet 10, or properties of other substances in the toilet 10. One or more sensors of the flow meter device 102 may be located near a window 23 through which the properties are detected. Additional, different, or fewer components may be included.

The toilet seat 105 may include one or more features to align the one of more sensors of the flow meter device 102 and the window with the toilet bowl 23, or more specifically, with a specific, predetermined point in the toilet 10. For example, an inner region 117 of the seat 105 is configured to support the seat 105 at a raised angle so that the sensors of the flow meter device 102 are aligned with the toilet 10 when the seat 105 rests on the rim of the toilet 10. When the seat 105 is raised, the seat 105 may also rest against the lid so that the one or more sensors of the flow meter device 102 are aligned with the toilet 10. In addition or in the alternative, a spacer 120 may position the seat 105 at a predetermined angle when the seat 105 rests on the rim of the toilet 10 so that the one or more sensors of the flow meter device 102 are aligned with the toilet 10.

The one or more sensors of the flow meter device 102 may detect properties of the ambient environment, properties of water in the toilet 10, properties of urine in the toilet 10, or properties of other substances in the toilet 10.

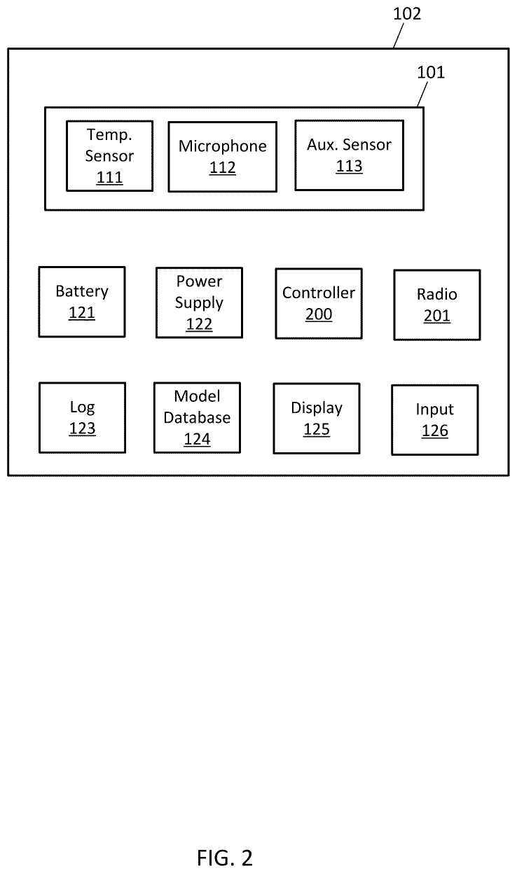

FIG. 2 illustrates an example block diagram for the flow meter device 102 of any embodiment. The flow meter device 102 may include a sensor array 101 and a controller 200. The sensor array 101 may include a temperature sensor 111, a microphone 112, and an auxiliary sensor 113. The sensor array 101 may include the temperature sensor 111 only, or the temperature sensor 111 and the auxiliary sensor 113. The flow meter device 102 may include input and output devices such as a display 125 and input 126. Additional, different, or fewer components may be included.

The microphone 112 may be configured to detect an audio (e.g., sound waves) from the urine flow. The microphone 112 may include a diaphragm configured to vibrate under forces received from sound waves. The diaphragm may move a coil that magnetically generates an electrical signal in a wire or trace. The electrical signal is proportional to the audio from the urine flow. The electrical signal may be provided to the controller 200. The electrical signal may be filtered and/or sampled before sending to the controller 200. The microphone 112 may be omitted.

The temperature sensor 111 is configured to detect a temperature in a toilet bowl associated with the urine flow. The temperature sensor 111 is a non-contact sensor. The temperature sensor 111 may include thermocouples or thermopiles configured to detect or absorb radiation from heat emitted by the urine and water in the toilet bowl. The temperature sensor 111 may be configured to measure a temperature of a surface of the fluid (e.g., water or water and urine) in the toilet bowl. The temperature of the surface of the fluid may be representative of the temperature (average temperature) of the fluid or the surface temperature may be proportional (related by a fraction or ratio) to the average temperature of the fluid. The temperature sensor 111 may include multiple temperature sensors, which may be mounted in a cluster or may be dispersed across multiple locations, such as multiple locations along the toilet seat 105. Specifically, the energy of the infrared light of the temperature sensor 111 is proportional to the water temperature.

The optional auxiliary sensor 113 may include another biometric sensor configured to detect the contents in the bowl. The auxiliary sensor 113 may be configured to detect the identity of the user. The auxiliary sensor 113 may detect one or more properties of the ambient environment. These examples are described in more detail below.

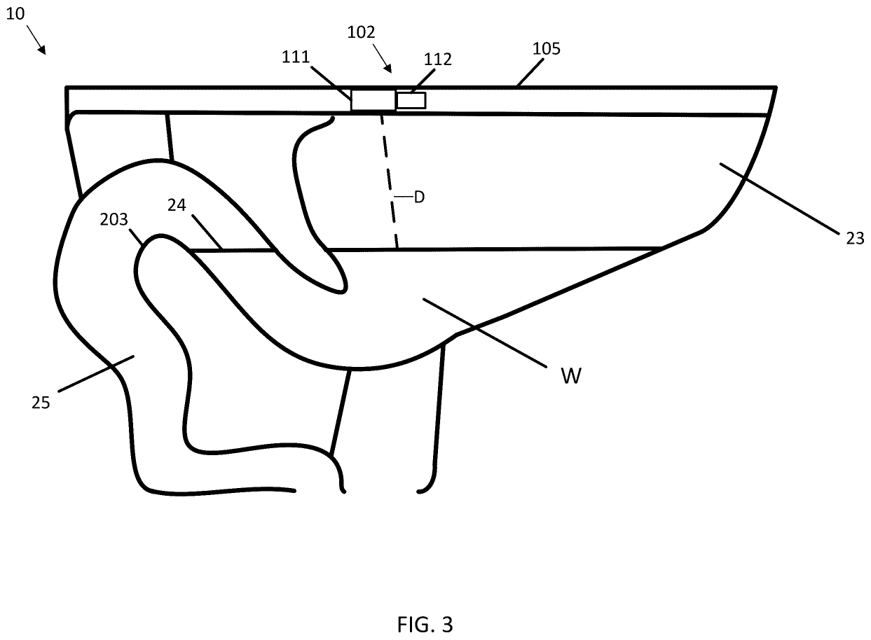

FIG. 3 illustrates a second embodiment of a flow meter device for a toilet 10 including a toilet bowl 23 in fluid communication with a trapway 25 defined by a dam 203. The height 24 for the bowl 23 and trapway 25 defines a distance from the flow meter device 102 to the water. In this example, the flow meter device 102 is located in a more forward position in the toilet seat 105. The flow meter device 102 is oriented to provide a line of sight D from the temperature sensor 111 to the water W in the toilet bowl 23. The microphone 112 may be positioned adjacent to the temperature sensor 111 or in another location, such as anywhere along the seat 105.

The controller 200 is configured to calculate a urine volume in the toilet bowl 23 based on the temperature in the toilet bowl. The controller 200 may select a bowl volume value and a bowl temperature value, for example, from pre-stored values in the model database 124. The bowl volume value may be set according to the type or model of the toilet 10. The bowl volume may be selected by the input 126. In another example, the bowl volume may be set according to another property for the toilet 10 such as a trapway height 24 for the trapway 25 or a float height in the tank.

The bowl temperature value may be the steady state or ambient temperature of the toilet 10. The bowl temperature value may be the long term average recorded by the temperature sensor 111. For example, the bowl temperature value may be the temperature average over a day, a month, or other time period. Alternatively, the bowl temperature value may be entered by the user at input 126. In another example, the bowl temperature value may be an ambient temperature in the room and may be received wirelessly from a thermostat.

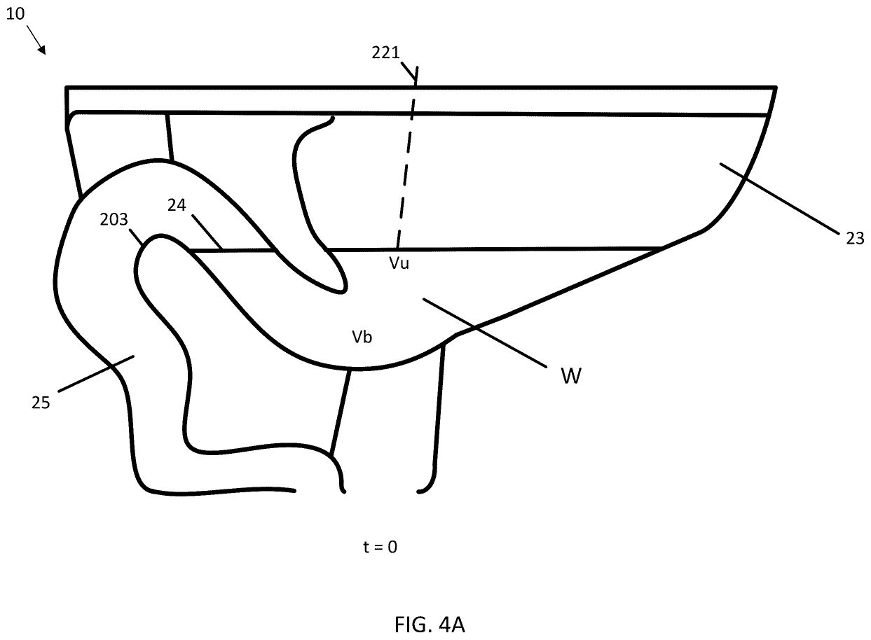

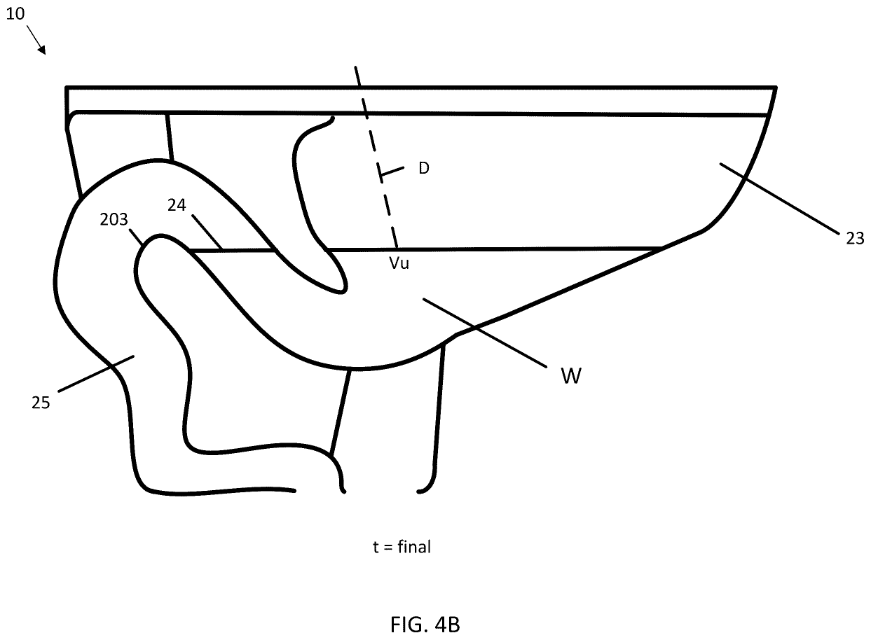

FIGS. 4A and B illustrates an example uroflowmeter calculation for the flow meter device. FIG. 4A illustrates the change in volume of urine in the bowl, and as a result, the change in the ratio of water to urine in the bowl. Initially, as shown by t=0 representing an initial time, the bowl water will be substantially the ambient temperature of the room or the temperature of the water supply. As urine stream 221 is provided to the bowl, the water level against the dam 203 of the trapway may rise. In some examples, some spill over the dam 203 is expected and may be accounted for in the calculations described herein with a spillover term.

As shown in FIG. 4B, the urine becomes a greater percentage of the contents of the bowl, which raises the overall temperature of the bowl. The temperature will increase proportionally to the amount of urine that is added to the bowl. In some examples, the temperature may be monitored over time. In other examples, the final temperature of the water and urine is measured at a time t=final.

The final time may be determined in a variety of techniques. In one example, the temperature reading is deemed final when the temperature does not substantially change for a predetermined number of readings or for a predetermined amount of time. In other examples, the temperature reading is taken and/or deemed final in response to a flush command or another aspect of the flush cycle. In another example, the temperature is monitored substantially continuously and the final time is selected after the flush has occurred, which may be indicated on a graph of the temperature by a sharp decrease in temperature. In another example, the final time is indicated by the audio from the microphone 112 falling below a threshold value. In addition, the final time may depend on when the water and urine have had sufficient time to mix together. This may be a configurable and/or preset amount of time after the audio from the microphone 112 has fallen below a threshold value.

The controller 200 determines the urine volume based on the bowl volume value, the bowl temperature value, and the measured final temperature of the water and urine in the bowl as measured by the temperature sensor 111. Equation 1 describes an example calculation for the controller 200 to determine the urine volume Vᵤ.

$${V}_{u} = {V}_{b} \frac{{T}_{f} - {T}_{i}}{{T}_{u} - {T}_{f}} \qquad Equation 1$$

In equation 1, Vᵤ represents the urine volume, V_{b} represents the bowl volume value, T_{f} represents a final temperature of the water and the urine, Tᵢ represents an initial water temperature, and Tᵤ represents a temperature of the urine. The temperature of the urine may be assumed to be the body temperature of the human. The temperature of the urine may be selectable. Additional terms may be included in Equation 1. An error term, a spillover term, an evaporation term, or other terms may be added to or subtracted from the urine volume.

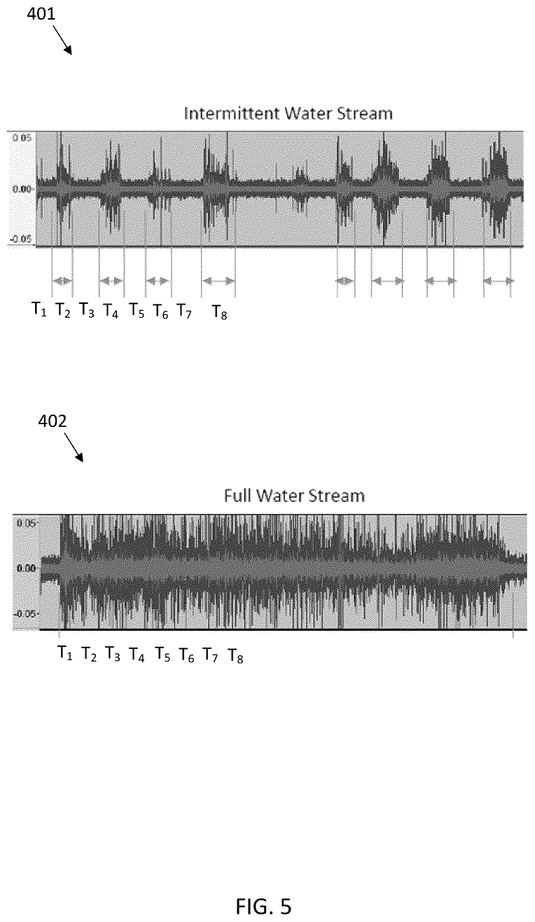

The controller 200 may calculate a flow rate for the user based on the urine volume from Equation 1 and at least one time interval from the audio from the urine flow. FIG. 5 illustrates an example sonourflowmeter calculation for the flow meter device. In chart 401, the audio output of an intermittent urine stream is illustrated, and in chart 402 the audio output of a continuous urine stream is illustrated.

The controller 200 is configured to identify at least one time interval based on a threshold level of the audio. For example, at each sample of the audio data, the controller 200 may compare the audio signal from the microphone 112 to a threshold level. For the time intervals where the samples that exceed the threshold, the controller 200 determines that urine is being provided to the bowl.

The controller 200 may generate a data array for the time interval that illustrates when the urine is being provided. In some examples, the array may include a binary value (i.e., either urine is being provided [1] or it is not [0]). The intermitted flow of chart 401 may be represented by Table 2. Even time intervals may be used (e.g., the same sampling rate).

TABLE 1

| TT₁ | T₂ | T₃ | T₄ | T₅ | T₆ | T₇ | T₈ |
|---|---|---|---|---|---|---|---|
| 0 | 1 | 0 | 1 | 0 | 1 | 0 | 1 |

Table 2 represents an example array for chart 402 where there is a continuation flow of urine, and for example, each sample meets or exceeds the audio signal threshold.

TABLE 2

| T₁ | T₂ | T₃ | T₄ | T₅ | T₆ | T₇ | T₈ |
|---|---|---|---|---|---|---|---|
| 1 | 1 | 1 | 1 | 1 | 1 | 1 | 1 |

Rather than the binary coefficient (e.g., 0 or 1), a variable coefficient may be used to represent various ranges of the audio signal for each of the time intervals in the array.

When the time intervals have the same duration, the coefficients may be summed to calculate the total duration of the urine stream (e.g., number of time intervals multiplied by the time interval duration). When the time intervals are variable, the individual durations of nonzero coefficients may be summed in a similar manner. The controller 200 may calculate the urine flow rate (Q) based on the volume of the urine (Equation 1, Vᵤ) and the total duration of the urination stream Tₜₒₜₐₗ as shown in Equation 2.

$$Q = \frac{{V}_{u}}{{T}_{total}} \qquad Equation 2$$

Additional terms may be included in Equation 2 to account for spillover, errors, evaporation, or other factors that impact the flow rate. In addition, a factor or other term may be added for the volume of the bowl as determined by the model of the toilet 10.

The controller 200 may be configured to store any of these data in the memory (e.g., log 123). The log 123 may include a timestamp stored in associated with the flow rate of the urine volume for a visit to the toilet 10 by a particular user of the toilet 10. The log 123 may also include an identifier for the user.

The controller 200 may identify the user with a variety of techniques. The input 126 may include a “on” or “log” button that the user depresses to instruct the controller 200 to record and store data for the visit to the toilet 10. In this example, only one user's data is logged, but others may still use the toilet. In another example, the user may enter a user ID into the input 126.

In another example, the auxiliary sensor 113 may detect and/or identify the user based on fingerprints, a heat signature, or an image (e.g., face recognition, body recognition). The auxiliary sensor 113 or the microphone 112 may detect a voice of the user. The auxiliary sensor 113 may detect the user based on radio frequency identification (RFID) or near field communication (NFC). The radio 201 may identify a mobile device of the user using Bluetooth or another wireless signal to determine the identifier of the user.

The controller 200 may calculate, output, and/or log other properties of the urine stream. For example, the controller 200 may determine a maximum rate of the urine flow based on the rate of change of temperature from the temperate sensor (e.g., derivative of temperature with respect to time). The controller 200 may calculate the time to maximum as the time interval up to the point where the maximum rate occurs. The controller 200 may determine the intermittency of the urine stream based on the sum of the time intervals with zero coefficients or otherwise having audio components below the threshold value.

The flow meter device 102 may include a battery 121 and/or a power supply 122. The battery 121 may provide power to the controller 200 and the sensor array 101. In some examples, the flow meter device 102 may be directly connected to an electrical outlet through the power supply 122 (e.g., the battery 121 may be omitted). In other examples, the battery 121 may be removable for charging.

The flow meter device 102 may communicate with one or more external devices using the radio 201. For example, the data of the log 123 may be sent to a mobile device of the user. The data of the log 123 may be sent to a wireless network and a remote device via a network connection and the internet. The data of the log 123 may be sent to a smart speaker or internet of things (IOT) device.

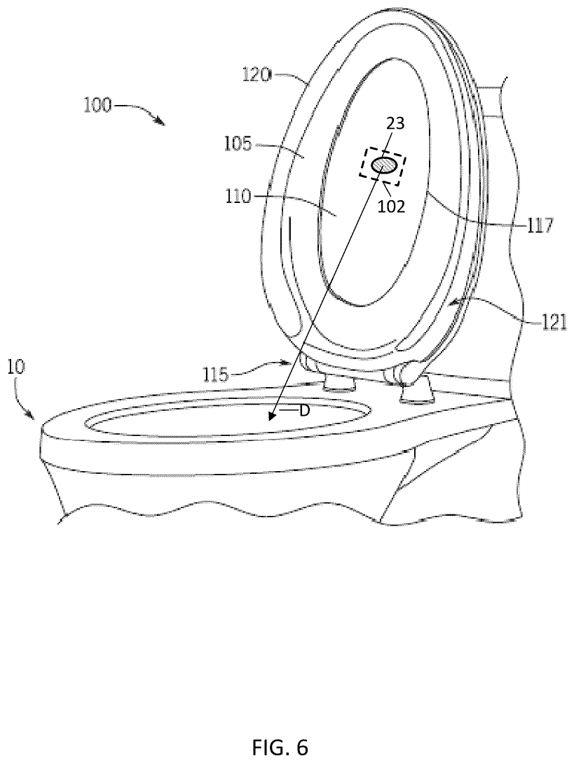

FIG. 6 illustrates a third embodiment of a flow meter device 102 for a toilet 10 wherein the microphone 112 and the temperature sensor 111 are mounted on a toilet lid 110. The window 23 may be oriented so that when the lid 110 is in a resting position, the line of sight D is pointed to a predetermined portion of the toilet bowl.

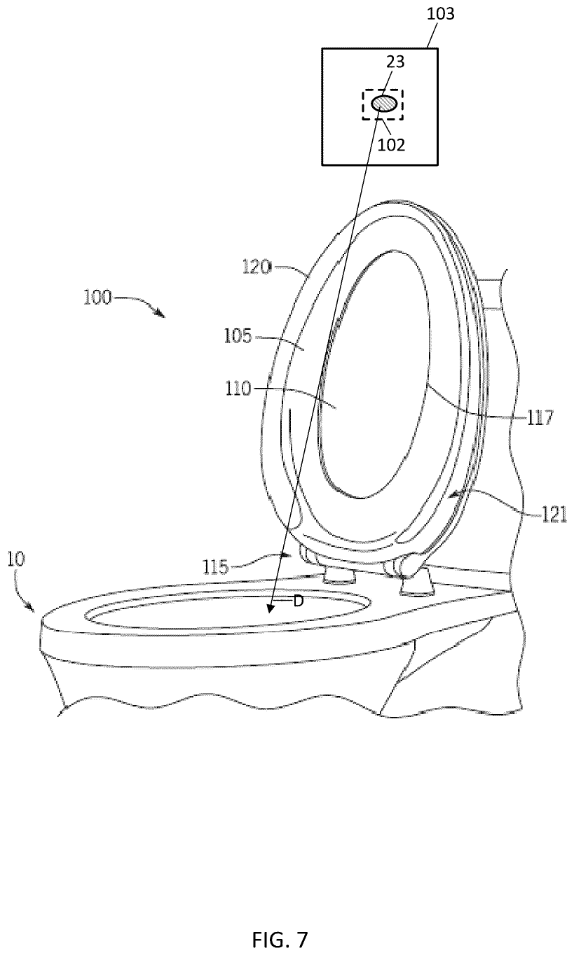

FIG. 7 illustrates a fourth embodiment of a flow meter device 102 for a toilet 10. A housing 103 for the flow meter device 102 is mounted on a wall adjacent to the toilet 10. The housing 103 may include one or more pivots to aim the line of sight D to the predetermined position of the toilet bowl.

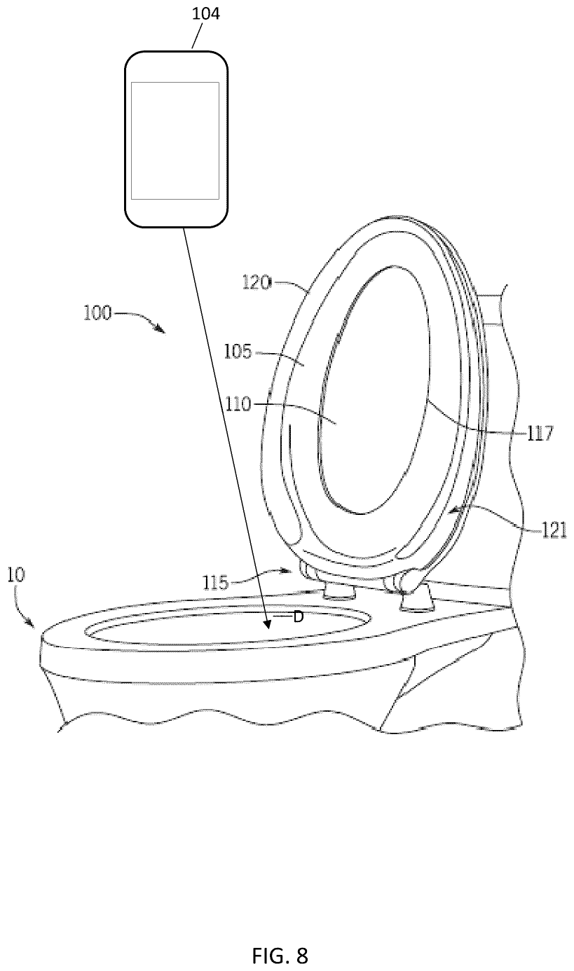

FIG. 8 illustrates a fifth embodiment of a flow meter device 104 for a toilet. In this example, a smartphone, a tablet, or another wireless communication device includes the microphone 112 and the temperature sensor 111. The user may place the wireless communication device in a holder mounted on the wall, on the toilet 10, on the toilet seat 105, or on the lid 110. The holder may orient the wireless communication device so that the line of sight D is aimed at the predetermined portion of the toilet bowl.

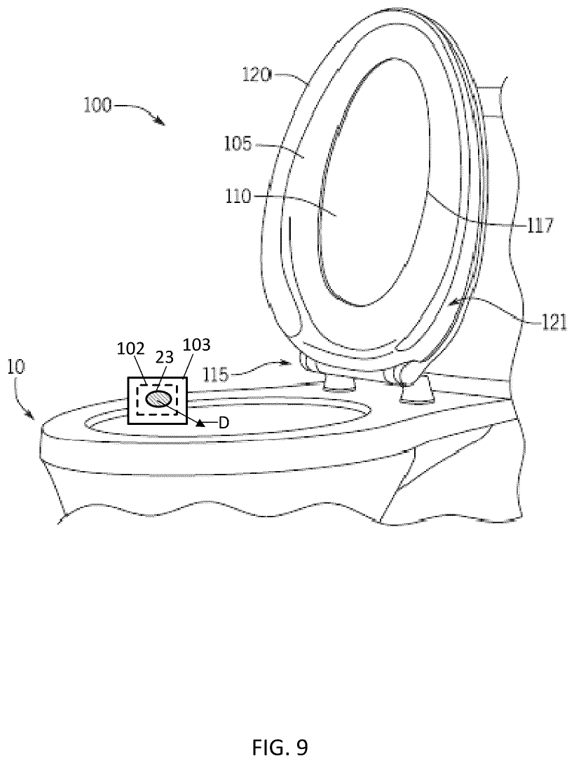

FIG. 9 illustrates a sixth embodiment of a flow meter device 102 for a toilet 10. In this example, the housing 103 is configured to rest on the rim to provide the flow meter device 102 and window 23 and the appropriate line of sight D to collect data at the predetermined position of the toilet bowl. The housing may clamp or otherwise be friction fit to the rim. In this example, the flow meter device 102 does not move when the seat 105 or lid 110 is raised or lowered.

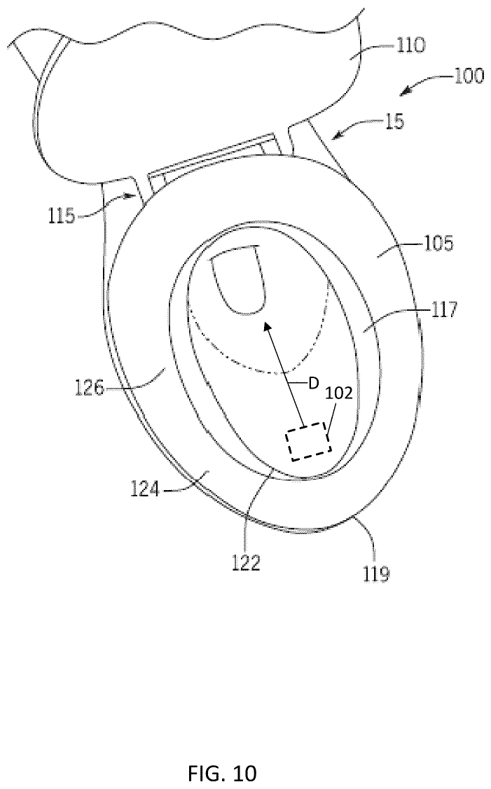

FIG. 10 illustrates a seventh embodiment of a flow meter device 102 for a toilet 10. One or more of the microphone 112 and the temperature sensor 111 may be mounted in the vitreous of the toilet bowl. In one example, the microphone 112 and the temperature sensor 111 may be placed in a cavity (e.g., formed from an insert mold). A window may be placed over the microphone 112 and the temperature sensor 111. The window may be permeable to infrared light for the temperature sensor 111 and transmit sound for the microphone 112. The flow meter device 102 may include a battery such that the flow meter device 102 may be removed to charge and/or replace the battery.

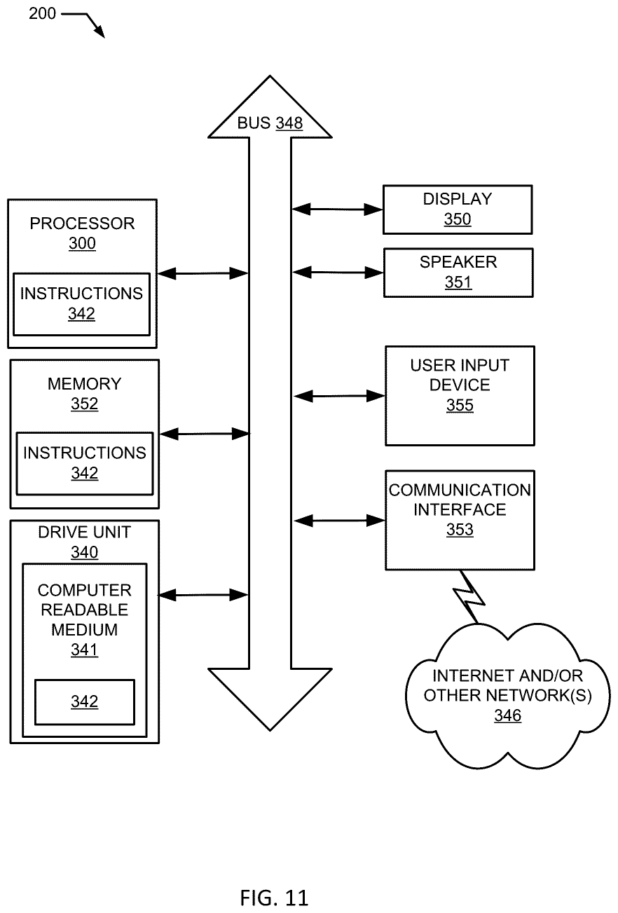

FIG. 11 illustrates an example controller for any of the disclosed embodiments. The controller 200 may include a processor 300, a memory 352, and a communication interface 353 for interfacing with devices or to the internet and/or other networks 346. In addition to the communication interface 353, a sensor interface may be configured to receive data from the sensors described herein or data from any source. The controller 200 may include an integrated display 350, speaker 351, or other output devices. The components of the control system may communicate using bus 348. The control system may be connected to a workstation or another external device (e.g., control panel) and/or a database for receiving user inputs, system characteristics, and any of the values described herein.

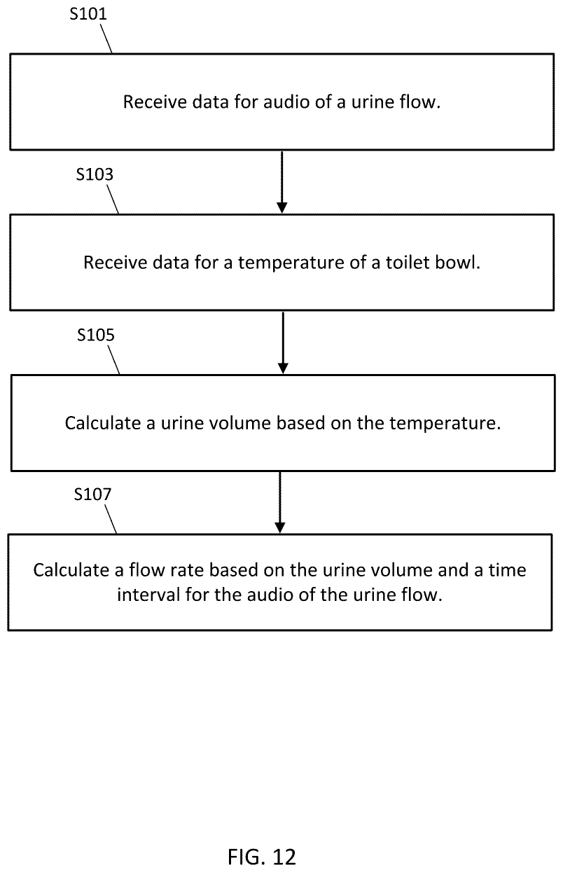

FIG. 12 illustrates an example flow chart for the operation of the flow detection device. Additional, different, or fewer acts may be performed.

At act S101, the processor 300 receives data for audio of a urine flow into a toilet bowl. The processor 300 may simplify the audio to audio data. The audio data may include a flag (e.g., ON or OFF) for each of a plurality of successive time intervals that indicates whether audio was collected during the respective time interval. The processor 300 may perform the derivative or integral of the audio data in order to simplify the audio data. The processor 300 may filter the audio to a predetermined frequency range in order to simplify the audio data.

At act S103, the processor 300 receives data for a temperature in the toilet bowl associated with the urine flow. The processor 300 may also simplify the temperature data. For example, the temperature may be sampled at a predetermined time interval. The temperature may be recorded as two significant digits. In addition, the temperature may be recorded as low, medium, or high or another set of classifications that simplifies the amount of data required for the temperature.

At act S105, the processor 300 calculates a urine volume in the toilet bowl based on the temperature in the toilet bowl. The processor 300 may determine a physical characteristics of the bowl based on an input received from the user input device 355 or a lookup table stored in the memory 352. The processor 300 compares the temperature to a predetermined scale of values based on the physical characteristics of the bowl, which identifies the urine volume in the bowl.

At act S107, the processor 300 calculates a flow rate based on the urine volume and at least one time interval from the audio from the urine flow. The processor 300 may calculate an average flow rate over time. The processor 300 may calculate a intermittency value for the urine stream.

Optionally, the control system may include an input device 355 and/or a sensing circuit 356 in communication with any of the sensors. The sensing circuit receives sensor measurements from sensors as described above. The input device may include any of the user inputs such as buttons, touchscreen, a keyboard, a microphone for voice inputs, a camera for gesture inputs, and/or another mechanism.

Optionally, the control system may include a drive unit 340 for receiving and reading non-transitory computer media 341 having instructions 342. Additional, different, or fewer components may be included. The processor 300 is configured to perform instructions 342 stored in memory 352 for executing the algorithms described herein. A display 350 may be an indicator or other screen output device. The display 350 may be combined with the user input device 355.

Processor 300 may be a general purpose or specific purpose processor, an application specific integrated circuit (ASIC), one or more programmable logic controllers (PLCs), one or more field programmable gate arrays (FPGAs), a group of processing components, or other suitable processing components. Processor 300 is configured to execute computer code or instructions stored in memory 352 or received from other computer readable media (e.g., embedded flash memory, local hard disk storage, local ROM, network storage, a remote server, etc.). The processor 300 may be a single device or combinations of devices, such as associated with a network, distributed processing, or cloud computing.

Memory 352 may include one or more devices (e.g., memory units, memory devices, storage devices, etc.) for storing data and/or computer code for completing and/or facilitating the various processes described in the present disclosure. Memory 352 may include random access memory (RAM), read-only memory (ROM), hard drive storage, temporary storage, non-volatile memory, flash memory, optical memory, or any other suitable memory for storing software objects and/or computer instructions. Memory 352 may include database components, object code components, script components, or any other type of information structure for supporting the various activities and information structures described in the present disclosure. Memory 352 may be communicably connected to processor 300 via a processing circuit and may include computer code for executing (e.g., by processor 300) one or more processes described herein. For example, the memory 352 may include graphics, web pages, HTML files, XML files, script code, shower configuration files, or other resources for use in generating graphical user interfaces for display and/or for use in interpreting user interface inputs to make command, control, or communication decisions.

In addition to ingress ports and egress ports, the communication interface 353 may include any operable connection. An operable connection may be one in which signals, physical communications, and/or logical communications may be sent and/or received. An operable connection may include a physical interface, an electrical interface, and/or a data interface. The communication interface 353 may be connected to a network. The network may include wired networks (e.g., Ethernet), wireless networks, or combinations thereof. The wireless network may be a cellular telephone network, an 802.11, 802.16, 802.20, or WiMax network, a Bluetooth pairing of devices, or a Bluetooth mesh network. Further, the network may be a public network, such as the Internet, a private network, such as an intranet, or combinations thereof, and may utilize a variety of networking protocols now available or later developed including, but not limited to TCP/IP based networking protocols.

While the computer-readable medium (e.g., memory 352) is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein.

In a particular non-limiting, exemplary embodiment, the computer-readable medium can include a solid-state memory such as a memory card or other package that houses one or more non-volatile read-only memories. Further, the computer-readable medium can be a random access memory or other volatile re-writable memory. Additionally, the computer-readable medium can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored. The computer-readable medium may be non-transitory, which includes all tangible computer-readable media.

In an alternative embodiment, dedicated hardware implementations, such as application specific integrated circuits, programmable logic arrays and other hardware devices, can be constructed to implement one or more of the methods described herein. Applications that may include the apparatus and systems of various embodiments can broadly include a variety of electronic and computer systems. One or more embodiments described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

It is intended that the foregoing detailed description be regarded as illustrative rather than limiting and that it is understood that the following claims including all equivalents are intended to define the scope of the invention. The claims should not be read as limited to the described order or elements unless stated to that effect. Therefore, all embodiments that come within the scope and spirit of the following claims and equivalents thereto are claimed as the invention.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances, where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Further, unless otherwise noted, the use of the words “approximate,” “about,” “around,” “substantially,” etc., mean plus or minus ten percent.

We claim:

1. An apparatus for measurement of urine flow, the apparatus comprising:
a microphone configured to detect an audio from the urine flow;
a temperature sensor configured to detect a temperature in a toilet bowl associated with the urine flow; and
a controller configured to calculate a urine volume in the toilet bowl based on the temperature in the toilet bowl and calculate a flow rate based on the urine volume and at least one time interval from the audio from the urine flow.

2. The apparatus of claim 1, wherein the controller selects a bowl volume value and a bowl temperature value, wherein the urine volume is calculated based on the bowl volume value and the bowl temperature value.

3. The apparatus of claim 2, wherein the controller calculates the urine volume as:
$${V}_{u} = {V}_{b} \frac{{T}_{f} - {T}_{i}}{{T}_{u} - {T}_{f}}$$
wherein Vᵤ represents the urine volume, V_{b} represents the bowl volume value, T_{f} represents a final temperature of water and the urine, Tᵢ represents an initial water temperature, and Tᵤ represents a temperature of the urine.

4. The apparatus of claim 1, wherein the controller identifies the at least one time interval based on a threshold level of the audio.

5. The apparatus of claim 1, wherein the at least one time interval includes a plurality of time intervals defined in an array.

6. The apparatus of claim 5, wherein the controller identifies an acoustic coefficient for each of the plurality of time intervals in the array.

7. The apparatus of claim 1, further comprising:
a memory configured to store data for the urine volume in the toilet bowl.

8. The apparatus of claim 7, wherein the data for the urine volume or the urine flow rate is stored in association with a user identifier and a timestamp.

9. The apparatus of claim 1, further comprising:
a radio configured to communicate the urine volume or the urine flow rate.

10. The apparatus of claim 1, wherein the controller is configured to identify a user of the toilet and generate a user identifier.

11. The apparatus of claim 1, wherein the microphone and the temperature sensor are mounted within a toilet seat.

12. The apparatus of claim 1, wherein the microphone and the temperature sensor are mounted on a toilet lid.

13. The apparatus of claim 1, wherein the microphone and the temperature sensor are mounted within an external device or mounted on wall adjacent to the toilet.

14. The apparatus of claim 1, wherein the controller calculates the flow rate as:
$$Q = \frac{{V}_{u}}{T}$$
wherein Q represents the flow rate, Vᵤ represents the urine volume, and T represents the at least one time interval from the audio from the urine flow.

15. The apparatus of claim 14, wherein T represents a sum of a plurality of time intervals including the at least one time interval from the audio from the urine flow.

16. A toilet comprising:
a toilet bowl; and
a temperature sensor configured to detect a temperature in the toilet bowl,
wherein a urine volume in the toilet bowl is calculated based on the temperature in the toilet bowl.

17. The toilet of claim 16, wherein the temperature sensor is coupled to a toilet seat or a lid.

18. The toilet of claim 16, wherein the temperature sensor is configured to measure a surface of a fluid in the toilet bowl.

19. A method for determining urination flow rates, the method comprising:
receiving data for a temperature in the toilet bowl associated with the urine flow;
calculating a urine volume in the toilet bowl based on the temperature in the toilet bowl; and
calculating a flow rate based, at least in part, on the urine volume.

20. The method of claim 19, further comprising:
receiving data for audio of a urine flow into a toilet bowl, wherein the flow rate is based, at least in part, on at least one time interval from the audio of the urine flow.
